(19) United States
(12) Patent Application Publication
(10) Pub. No.: US 2016/0129217 A1
(43) Pub. Date: May 12, 2016

(54) HYPNOTIC SYSTEM AND METHOD FOR THE SAME

(71) Applicants: Inventec (Pudong) Technology Corporation, Shanghai (CN); INVENTEC CORPORATION, Taipei City (TW)

(72) Inventor: Feng-Wei HSU, Taipei City (TW)

(21) Appl. No.: 14/677,482

(22) Filed: Apr. 2, 2015

(30) Foreign Application Priority Data
Nov. 7, 2014 (CN) 201410625244.9

(51) Int. Cl. A61M 21/02; A61B 5/00; A61B 5/024; A61B 5/0476; A61B 5/0488

(52) U.S. Cl. CPC A61M 21/02; A61B 5/0476; A61B 5/0488; A61B 5/024; A61B 5/4812; A61M 2021/0027

(57) ABSTRACT

A hypnotic system includes a physiological parameter detection device, an audio information comparison device, a communication device and an audio device. The physiological parameter detection device detects the physiological parameter of a user for generating a physiological parameter signal. The audio information comparison device receives the physiological parameter signal, locates a respective audio information from an internal audio-comparing table, and then generates a corresponding audio signal for transmitting to the communication device. The audio device receives the audio signal forwarded by the communication device and then plays the corresponding audio information matching user's physiological parameter so as to serve a hypnotic purpose to the user.

### BACKGROUND OF INVENTION

1. Field of the Invention

The invention relates to a hypnotic system and a method for the same, and more particularly to the hypnotic system and method that judge user's physiological parameters to furnish adequate audio information for the user to generate a hypnotic environment.

2. Description of the Prior Art

As the modern technology progresses every day, people demand in a quality life has been gradually raised. Due to busy daily life and the rising living pressure, and also due to the bad habit of using the computer, the mobile phone, the tablet computer or any electronic product the like prior to the bedtime, the insomnia has become more and more common to ordinary people.

It is well known that people have spent ⅓ or ¼ of their life time for sleeping. Obviously, if the insomnia becomes a problem, the spirits and body health would be terribly influenced. Definitely, under such a circumstance, some clinical and unexpected illness would be inevitably true. Hence, health consciousness prevails in recent years. People have gradually paid more attention to their individual healthy states, and more and more relaxing methods have been introduced and developed. Some of these efforts are devoted to promoting the comfort toward a satisfied sleeping. However, while most of these hypnotic approaches are stressed on the audio information such as the music to help people fall sleepy, but the difficulty thereabout is the correct choice of relevant audio information individually. Often, people are hard to get sleepy for various reasons. Hence, how to develop an improved hypnotic system that can really help people to choose relevant audio waves for getting sleepy and relaxed physically and mentally is definitely welcome to the skilled persons in the art.

### SUMMARY OF THE INVENTION

Accordingly, in order to help people to determine a preferred audio waves or information for achieving hypnosis, the present invention provides a hypnotic system that can base on user's physiological parameters to furnish more adequate audio information to hypnotize the user.

In the present invention, the hypnotic system mainly includes a physiological parameter detection device, an audio information comparison device and a communication device. The physiological parameter detection device is used to detect at least one physiological parameter of a user so as thereby to generate and then transmit out a physiological parameter signal. The audio information comparison device includes a first transmission module, a comparison and storage module and a first processing module. The first transmission module is communicatively coupled with the physiological parameter detection device for receiving and then forwarding the physiological parameter signal. The comparison and storage module stores an audio-comparing table of physiological parameters, in which the audio-comparing table of physiological parameters includes a plurality of audio information. The first processing module electrically coupled with the first transmission module and the comparison and storage module is to receive the physiological parameter signal so as thereby to locate at least one audio information matching the physiological parameter of the user from the audio-comparing table of physiological parameters in the comparison and storage module. Based on the at least one matched audio information, a corresponding audio signal is generated by the first processing module and then transmitted out therefrom.

The communication device includes a second transmission module communicatively coupled with the first transmission module of the audio information comparison device and is to receive and then forward the audio signal. In the present invention, an audio device can be included to receive the audio signal. The audio device further includes a third transmission module and an audio module. The third transmission module communicatively coupled with the second transmission module of the communication device is to receive and then forward the audio signal transmitted from the second processing module. The audio module electrically coupled with the third transmission module is to receive the audio signal and then play the audio information.

In one embodiment of the present invention, the first processing module of the audio information comparison device can base on the physiological parameters to determine the sleep cycle information of the user, so that the audio information matching the physiological parameter can be located from the audio-comparing table of physiological parameters. In the present invention, the audio information can be at least one of a music and a natural sounds, and the physiological parameter can be at least one of a brain wave, a heartbeat, a breath and a myoelectricity.

In one embodiment of the present invention, the audio information comparison device can be a network server, the communication device can be one of a mobile phone and a tablet computer, and the audio device can be one of an earphone and a speaker.

In the present invention, the hypnotic method is to firstly detect at least one first physiological parameter of a user so as thereby to generate and then transmit a first physiological parameter signal. Then, the first physiological parameter signal is received so as to further locate at least one first audio information matching the first physiological parameter from an audio-comparing table of physiological parameters containing a plurality of first audio information. The first audio signal is then received so as further to play the corresponding first audio information. In addition, at least one second physiological parameter of the user is detected so as to generate and forward a second physiological parameter signal. Upon receiving the second physiological parameter signal, a corresponding sleep cycle information is judged so as thereby to locate at least one second audio information matching the second physiological parameter from the audio-comparing table of physiological parameters, such that a second audio signal can be generated and then transmitted. Finally, the second audio signal is received, and the corresponding second audio information is played.

In one embodiment of the present invention, the first audio information and the second audio information can be played simultaneously.

By providing the hypnotic system and the accompanying hypnotic method in accordance with the present invention, the physiological parameter of the user can be evaluated to determine adequate audio information to help the user to achieve hypnosis by playing the favorite audio information. By compared to the current art, the hypnotic system and method in accordance with the present invention is superior in helping the user to get sleepy.

All these objects are achieved by the hypnotic system and the method for the same described below.

### BRIEF DESCRIPTION OF THE DRAWINGS

The present invention will now be specified with reference to its preferred embodiment illustrated in the drawings, in which:

FIG. 1 demonstrates schematically a typical sleep cycle;

FIG. 2 shows the relationship between the sleep cycles and the sleep stages;

FIG. 3 demonstrates schematically an application of the hypnotic system in accordance with the present invention;

FIG. 4 is a block-diagram view of a first embodiment of the hypnotic system in accordance with the present invention;

FIG. 5 demonstrates an audio-comparing table of physiological parameters in accordance with the present invention;

FIG. 6 demonstrates another audio-comparing table of physiological parameters in accordance with the present invention;

FIG. 7 is a flowchart of a preferred embodiment of the hypnotic method in accordance with the present invention; and

FIG. 8 is a block-diagram view of a second embodiment of the hypnotic system in accordance with the present invention.

### DESCRIPTION OF THE PREFERRED EMBODIMENT

The invention disclosed herein is directed to a hypnotic system and a method for the same. In the following description, numerous details are set forth in order to provide a thorough understanding of the present invention. It will be appreciated by one skilled in the art that variations of these specific details are possible while still achieving the results of the present invention. In other instance, well-known components are not described in detail in order not to unnecessarily obscure the present invention.

For various embodiments can be organized for the hypnotic system and the method for the same in accordance with the present invention, only a typical and preferred embodiment is elucidated in details with sufficient drawings in the following description.

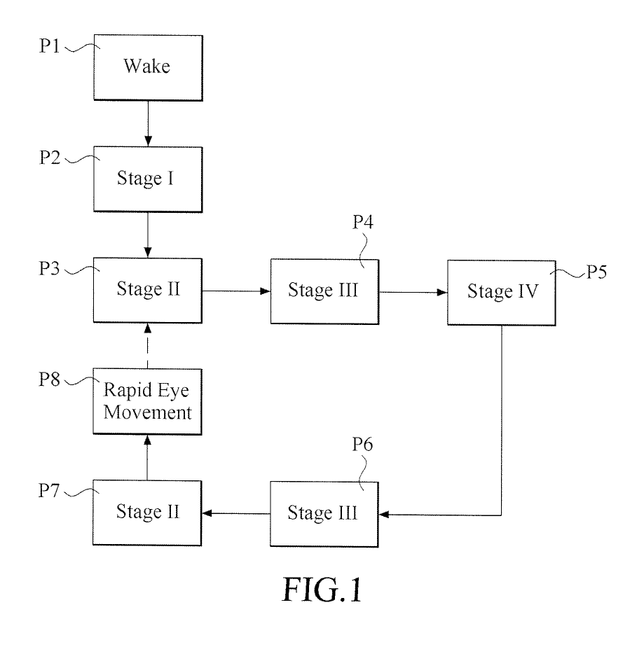

Referring now to FIG. 1, a typical sleep cycle is schematically demonstrated. As shown, to an ordinary person, about 20 minutes are needed to transit from a Wake stage P1 to a sleep state. The sleep state further includes a sub-state of rapid eye movement (REM) and another sub-state of non-rapid eye movement (NREM), in which the NREM sub-state can be further divided into Stage I P2, Stage II P3, Stage III P4 and Stage IV P5. Generally speaking, a sleep cycle (first sleep cycle) is said to sleep over in order the Stage I P2, the Stage II P3, the Stage III P4, the Stage IV P5, the Stage III P6, the Stage II P7 and the REM sub-state P8. Then, another sleep cycle begins at the REM sub-state P8, then the Stage II P3, and the same order as that in the first cycle). Normally, about 90 minutes are needed for a sleep cycle, and there are about 4-6 sleep cycles in a night.

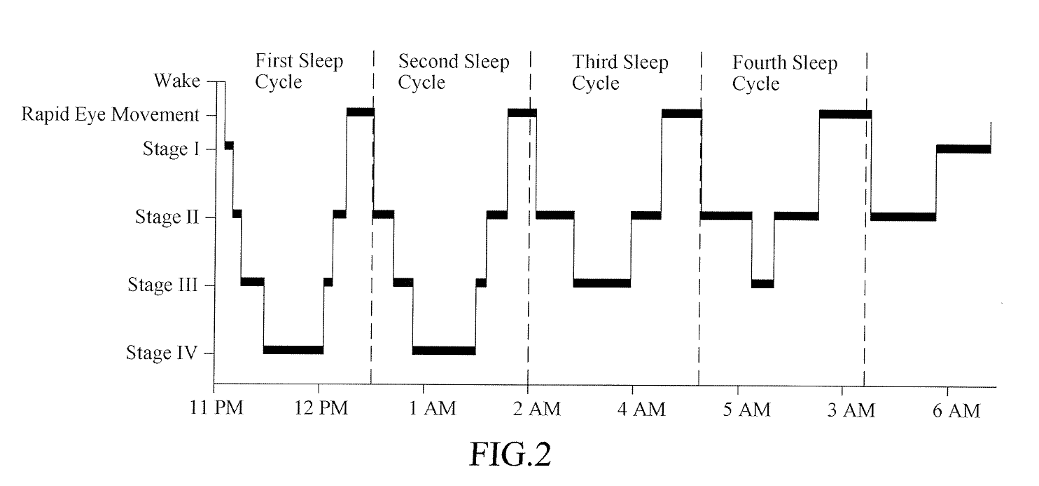

Referring now to FIG. 2, the relationship between the sleep cycles and the sleep stages is shown. As shown, the beginning stage of each sleep cycle might be slightly different. Yet, it is noted that the deep sleep stages, the Stage IV and the Stage III, are no more there in the sleep cycles close to the morning.

Therefore, to promote the sleep quality, either reducing the Wake stage P1 or repeating good sleep cycles shall be performed.

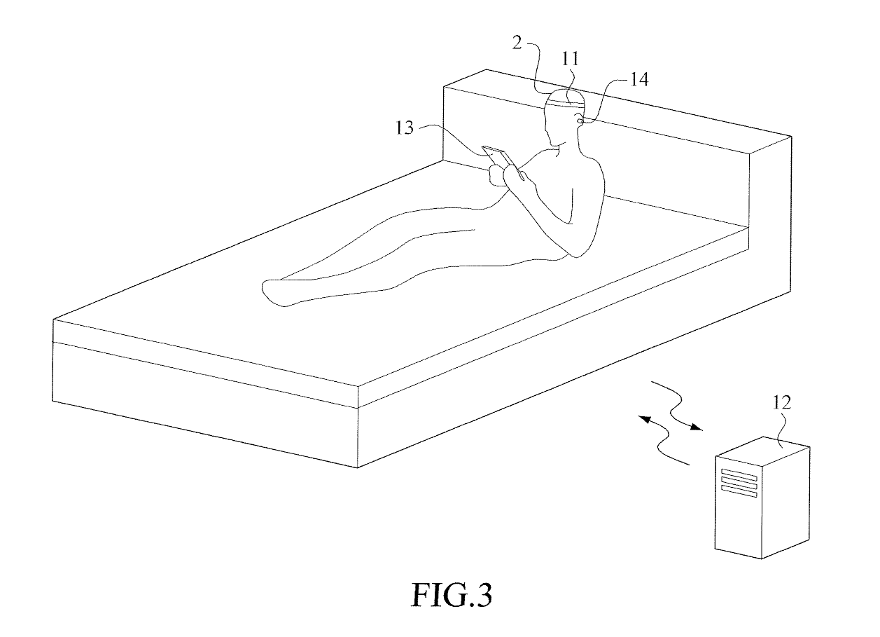

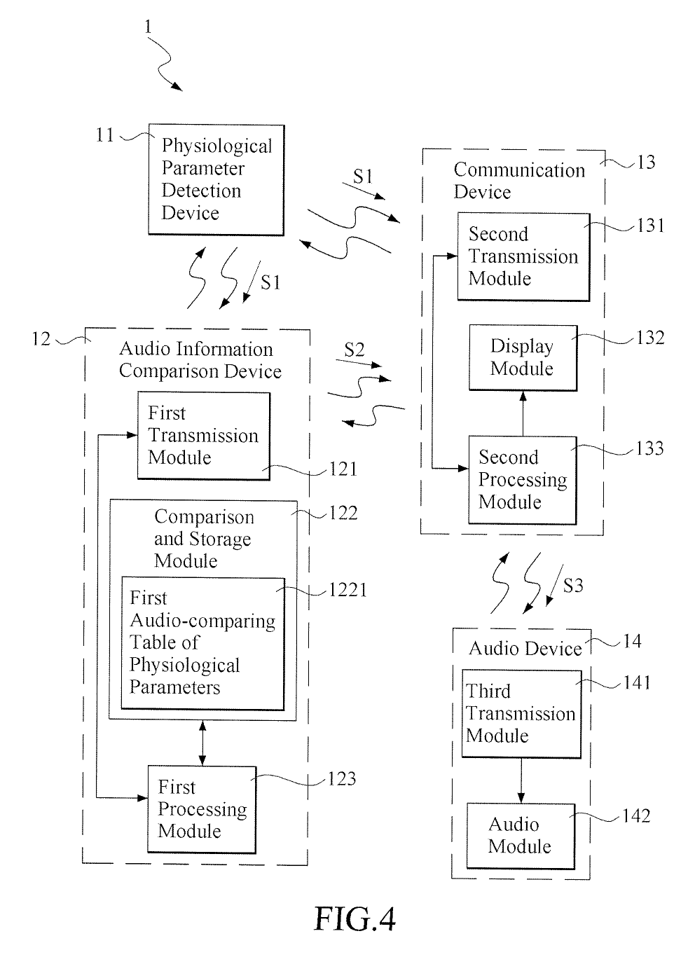

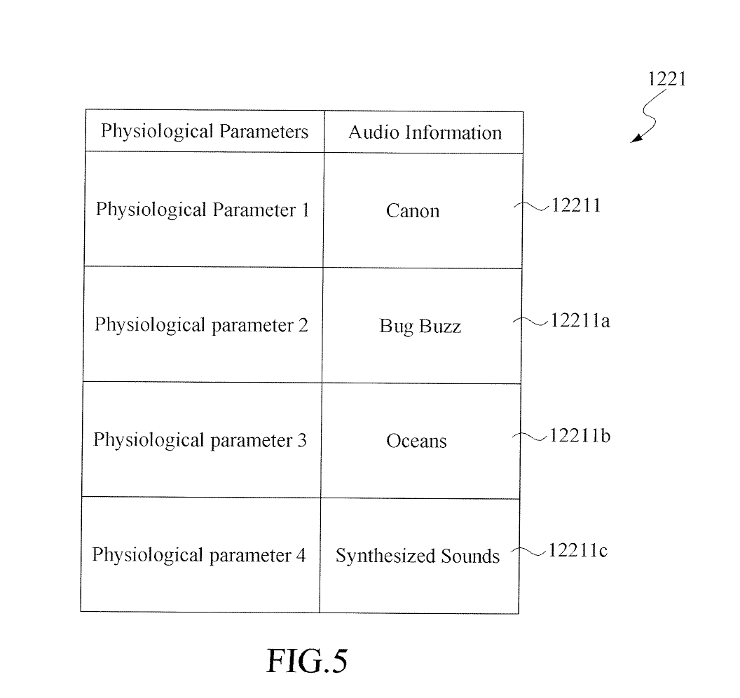

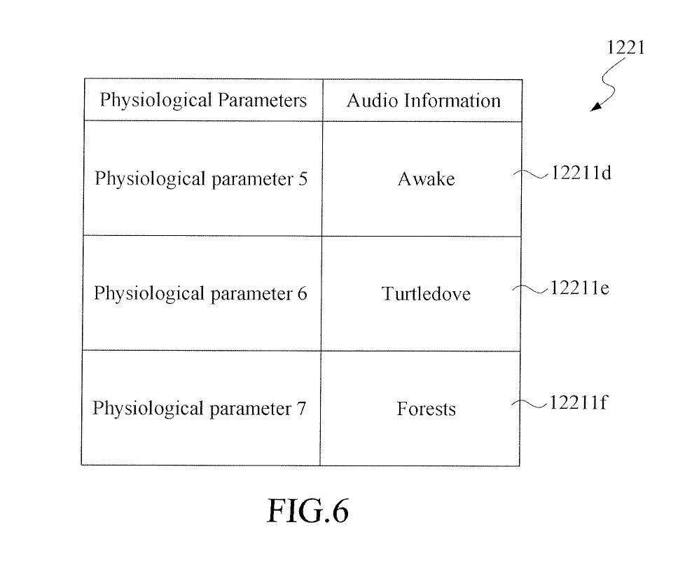

Refer now to FIG. 3 to FIG. 6, in which FIG. 3 demonstrates schematically an application of the hypnotic system in accordance with the present invention, FIG. 4 is a block-diagram view of a first embodiment of the hypnotic system in accordance with the present invention, FIG. 5 demonstrates an audio-comparing table of physiological parameters in accordance with the present invention, and FIG. 6 demonstrates another audio-comparing table of physiological parameters in accordance with the present invention.

The hypnotic system 1 of the present invention is provided to a user 2 who intend to sleep. The hypnotic system 1 includes a physiological parameter detection device 11, an audio information comparison device 12, a communication device 13 and an audio device 14.

The physiological parameter detection device 11 can be (but not limited to) a brain-wave detector, a heartbeat detector, a breath detector or a myoelectricity detector. In the preferred embodiment, the brain-wave detector is applied as the physiological parameter detection device 11. In addition, the physiological parameter detection device 11 can be equipped with a wireless communicatively coupling interface or a cable communicatively coupling interface. The wireless communicatively coupling interface can be a mobile communication network interface (such as 3G, LTE, WIMAX, or 5G), a bluetooth interface, a zigbee interface, a wireless fidelity (Wi-Fi) interface, or a radio frequency interface, and the cable communicatively coupling interface can be a universal series bus (USB) interface, a thunderbolt interface or an IEEE 1394 interface.

Further, the physiological parameter detection device 11 can be mounted to any portion (the head, the ear, the heart or the like) of a user 2, or any place near the user 2. For the physiological parameter detection device 11 of this preferred embodiment is a brain-wave detector, the physiological parameter detection device 11 herein is mounted to the head of the user 2.

The audio information comparison device 12 is communicatively coupled with the physiological parameter detection device 11, and can be an ordinary network server (or said a cloud-end server, or a far-end server). The audio information comparison device 12 includes a first transmission module 121, a comparison and storage module 122 and a first processing module 123. The first transmission module 121(a wireless communicative interface for example) is communicatively coupled with the wireless communicative interface of the physiological parameter detection device 11. The comparison and storage module 122 can be a non-volatile memory or a volatile memory. The first processing module 123 electrically coupled with the first transmission module 121 and the comparison and storage module 122 can be a central process unit (CPU), a graphic process unit (GPU) or an accelerated processing unit (APU).

The comparison and storage module 122 stores a audio-comparing table of physiological parameters 1221, and the audio-comparing table of physiological parameters 1221 includes a plurality of first audio information 12211, 12211a, 12211b, 12211c (as shown in FIG. 5, the Canon, the Bug buzz, the Oceans, the synthesized sounds, respectively). Furthermore, as shown in FIG. 6, the audio-comparing table of physiological parameters 1221 can further includes a plurality of second audio information 12211d, 12211e, 12211f (Awake, Turtledove, Forests, respectively).

It shall be noted that each of the first audio information 12211, 12211a, 12211b, 12211c and the second audio information 12211d, 12212e, 12212f can be a music, a natural sound, a synthesized wave or a combination thereof. As shown in FIG. 5, the first audio information 12211 and second audio information 12211d are music, the first audio information 12211a, 12211b and the second audio information 12212e, 12212f are all natural sounds, and the first audio information 12211c is a synthesized wave.

Further, in the present invention, the music herein can be a popular music or a classic music, and can be a complete music chapter or simply a small music section. The natural sounds can be defined as sounds of Forests, Oceans, Bug buzz, Bird cries, Winds and the like natural sounds. The synthesized waves are synthesized sounds, such as the Alpha wave or the Beta wave.

The communication device 13 communicatively coupled with the physiological parameter detection device 11 and the audio information comparison device 12 can be (but not limited to) a smart mobile phone or a tablet computer. The communication device 13 further includes a second transmission module 131, a display module 132 and a second processing module 133. The second transmission module 131 can be any of the aforesaid wireless communicative interfaces, and is communicatively coupled with the wireless communicative interface of the physiological parameter detection device 11 and the first transmission module 121 of the audio information comparison device 12. The display module 132 can be a monitor or a display screen. The second processing module 133 electrically coupled with the second transmission module 131 and the display module 132 can be a CPU, a GPU or an APU.

The audio device 14 mounted close to the user 2 or at the ear of the user 2 can be a speaker or an earphone, and is cable or wireless coupled with the communication device 13. In one embodiment of the present invention, the audio device 14 wirelessly coupled to the communication device 13 includes a third transmission module 141 and an audio module 142. The third transmission module 141 communicatively coupled with the second transmission module 131 can be any of the aforesaid wireless communicative interfaces and cable communicative interfaces. The audio module 142 electrically coupled with the third transmission module 141 can be formed as a unit piece.

Further, it shall be noted that, though communication device 13, the physiological parameter detection device 11 and the audio device 14 in the preferred embodiment are discrete devices, yet, in some other embodiments, the communication device 13 can be integrated to the audio device 14 so to form a unique device, or the physiological parameter detection device 11 can be integrated to the audio device 14 so as to form another unique device.

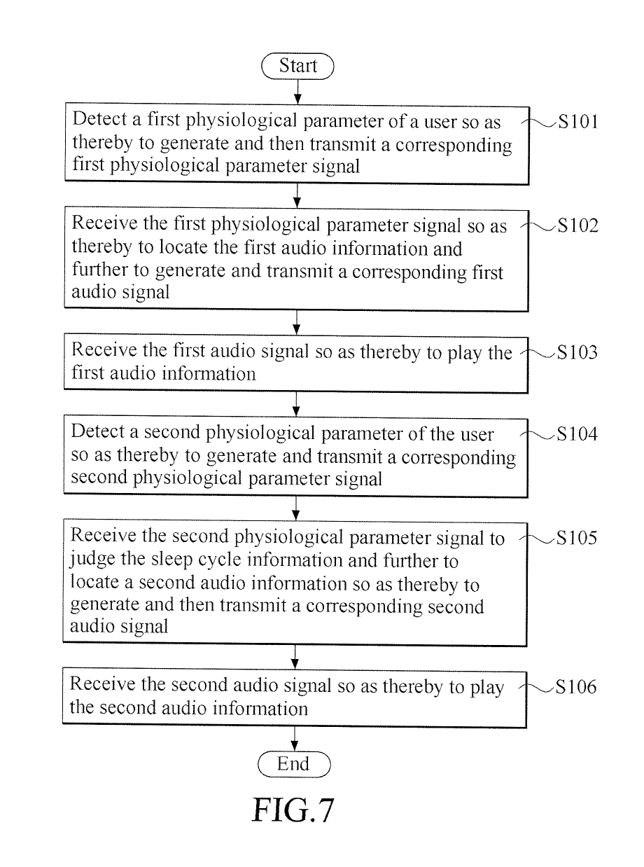

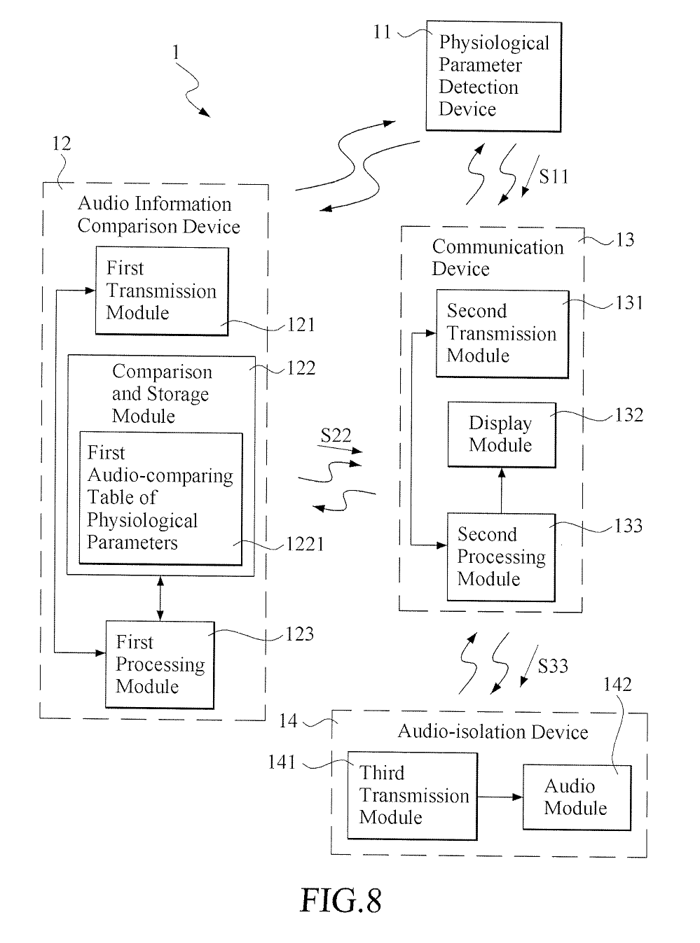

In order to further elucidate the application of the present invention, refer now to FIG. 7 and FIG. 8, in which FIG. 7 is a flowchart of a preferred embodiment of the hypnotic method in accordance with the present invention, and FIG. 8 is a block-diagram view of a second embodiment of the hypnotic system in accordance with the present invention.

As shown, the preferred embodiment of the hypnotic method in accordance with the present invention includes the following steps.

Step S101: Detect a first physiological parameter of a user so as thereby to generate and then transmit a corresponding first physiological parameter signal.

Step S102: Receive the first physiological parameter signal so as thereby to locate the first audio information and further to generate and transmit a corresponding first audio signal.

Step S103: Receive the first audio signal so as thereby to play the first audio information.

Step S104: Detect a second physiological parameter of the user so as thereby to generate and transmit a corresponding second physiological parameter signal.

Step S105: Receive the second physiological parameter signal to judge the sleep cycle information and further to locate a second audio information so as thereby to generate and then transmit a corresponding second audio signal.

Step S106: Receive the second audio signal so as thereby to play the second audio information.

While the user is ready to sleep, he/she can wear or properly locate the physiological parameter detection device 11 and the audio device 14. Then, start the application program (APP) of the communication device 13 by (but not limited by) touching the preset interface on the display module 132, such that the Step S101 can be started to detect at least one first physiological parameter of a user so as thereby to generate and then transmit a corresponding first physiological parameter signal. In this operation, while the APP is started, the physiological parameter detection device 11 is then initiated to detect at least one first physiological parameter of the user 2 so as thereby to generate and then transmit a corresponding first physiological parameter signal S1. In other embodiments, the physiological parameter detection device 11 can be triggered automatically at the same time of wearing the physiological parameter detection device 11. For the detection target of this embodiment is the brain wave of the user 2, the physiological parameter detection device 11 is a brain-wave detector. However, in other embodiments, the detection target can be a heartbeat, a breath or a myoelectricity. In addition, the first physiological parameter captured by the physiological parameter detection device 11 in this embodiment can be the raw information (brain waveform) of the brain wave or the sorted information from the raw information.

After the physiological parameter detection device 11 transmits the first physiological parameter signal S1, the step S102 is immediately performed. In the Step S02, the first transmission module 121 of the audio information comparison device 12 receives the first physiological parameter signal S1 from the second transmission module 131 of the communication device 13. After the first transmission module 121 receives the first physiological parameter signal S1, the first physiological parameter signal S1 is forwarded to the first processing module 123 so as thereby to locate at least one first audio information matching the first physiological parameter of the user 2 from the audio-comparing table of physiological parameters 1221 stored in the comparison and storage module 122. Based on the at least one first audio information, a first audio signal S2 is generated and then transmitted. In the present invention, the audio-comparing table of physiological parameters 1221 can be a lookup table for the first physiological parameter signal S1 and the first audio information.

In practice, the first processing module 123 realizes the first physiological parameter in the first physiological parameter signal S1 and thus bases on the first physiological parameter to locate the first audio information 12211 from the audio-comparing table of physiological parameters 1221. Hence, the first audio information 12211 is obtained by referring to the first physiological parameter signal S1 in a consideration of hypnosis. The first processing module 123 can look up the respective first audio information 12211 according to the first physiological parameter signal S1 in the audio-comparing table of physiological parameters 1221, and further, based on the first audio information 12211, the first audio signal S2 is formed and then forwarded. For example, if the first physiological parameter is a specific brain wave, the audio-comparing table of physiological parameters 1221 would list various types of brain waves and the corresponding first audio information 12211. In addition, the first physiological parameter can be sorted information of the detected brain wave. In such a circumstance, the first processing module 123 needs to refer the sorted information to look up the adequate first audio information 12211.

Then, perform the Step S103. The first processing module 123 transmits the first audio signal S2 to the communication device 13 via the first transmission module 121 and the second transmission module 131. The communication device 13 then transmits the first audio signal S2 in a form of a second audio signal S3 to the audio module 142 of the audio device 14 via the second transmission module 131 and the third transmission module 141 so as there to play the first audio information 12211. In other embodiments, the audio module 142 may be built in the communication device 13, and so the communication device 13 can directly play the first audio information 12211 for the user 2 to fall asleep.

After the user 2 sleeps, if a good sleep cycle can be repeated, the sleep quality can thus be assured. Further, prior to a preset wakeup time, the user 2 can gradually reduce his/her depth of sleep, such that a comfortable wakeup for the user 2 can be achieved. By providing the present invention, adequate audio information can be furnished to hypnotize the user 2 into better sleep cycles. For example, while in meeting notorious vehicle noises or shadows frequently in nights to hurt the sleep quality of the user 2, then the α wave, the β wave or the δ wave can be provided to help the users to gain back the depth of sleep, such that the good sleep cycle can be kept.

Hence, after the user 2 sleeps for a period of time, the Step 105 is further performed. Then, the physiological parameter detection device 11 detects at least one second physiological parameter of the user 2 and thereby generates a second physiological parameter signal S11 for transmission to the communication device 13. The communication device 13 would record and analyze the sleep information of the user 2. In the case that the user 2 is in a bad sleep cycle (for example, staying the REM state while in the Stage III), then the second processing module 133 of the communication device 13 would transmit the instant second physiological parameter signal S11 to the first transmission module 121 of the audio information comparison device 12 via the second transmission module 131. After the first transmission module 121 receives the second physiological parameter signal S11, the second physiological parameter signal S11 is further forwarded to the first processing module 123, and there a second audio information 12211d matching the second physiological parameter of the user 2 can be located from the audio-comparing table of physiological parameters 1221 stored in the comparison and storage module 122. Based on the second audio information 12211d, a third audio signal S22 can be generated and then transmitted. In the present invention, the audio-comparing table of physiological parameters 1221 can be a lookup table for the second physiological parameter signal S11 and the second audio information 12211d.

In other embodiments, the first processing module 123 can judge the sleep time to calculate the respective sleep cycle information for integrating the second physiological parameter signal S11 to locate the second audio information 12211d in the first audio-comparing table of physiological parameters 1221. In another embodiment, the first processing module 123 can analyze the second physiological parameter signal S11 to obtain the sleep cycle information, and then locate the second audio information 12211d in the first audio-comparing table of physiological parameters 1221 according to the sleep cycle information.

In this embodiment, the second audio information 12211d is generated by correlating the second physiological parameter signal S11 for maintaining user's good sleep cycles in the audio-comparing table of physiological parameters 1221. Namely, the first processing module 123 can judge the second physiological parameter signal S11 to form the second audio information 12211d and further to generate the third audio signal S22 for transmission.

Then, the Step S106 is performed. The first processing module 123 transmit the third audio signal S22 to the communication device 13 via the first transmission module 121 and the second transmission module 131. The communication device 13 further forward the third audio signal S22 in a form of a fourth audio signal S33 to the audio module 142 of the audio device 14 via the second transmission module 131 and the third transmission module 141 so as there to play the second audio information 12211d. In other embodiments, the audio module 142 can be built in the communication device 13, and thus the communication device 13 can directly play the second audio information 12211d to hypnotize the user 2. In some other embodiments, the audio module 142 can play the first audio information 12211 and the second audio information 12211d simultaneously.

Further, in another embodiment, the first processing module 123 can real-timely analyze the second physiological parameter of the user 2 and thus realize the sleep cycle information (the information shown in FIG. 1 and FIG. 2). Then, from the audio-comparing table of physiological parameters 1221, the second audio information 12211e can be obtained by referring to the corresponding physiological parameter signal S11. The second audio information 12211e is then forwarded to the audio module 142 for playing via the first transmission module 121, the second transmission module 131 and the third transmission module 141. Thus, at this time, the user 2 can be hypnotized by a mixed sound of the second audio information 12211d and the second audio information 12211e.

In addition, in another embodiment, the second processing module 133 can real-timely analyze the first physiological parameter or the second physiological parameter (while the physiological parameter detection device 11 forwards signals directly to the communication device 130) to realize the sleep cycle information of the user 2 for being transmitted to the audio information comparison device 12. Namely, the sleep cycle information can be realized either by the communication device 13 or by the audio information comparison device 12.

In summary, by providing the hypnotic system and method to help people in determining a preferred audio waves or information for achieving hypnosis according to user's personal and instant physiological parameter, a better sleep quality than ever can thus be easily achieved over most of hypnotic techniques in the art.

While the present invention has been particularly shown and described with reference to a preferred embodiment, it will be understood by those skilled in the art that various changes in form and detail may be without departing from the spirit and scope of the present invention.

What is claimed is:

1. A hypnotic system, comprising:
a physiological parameter detection device for detecting at least one physiological parameter of a user so as thereby to generate and then forward a physiological parameter signal;
an audio information comparison device, further comprising:
a first transmission module, communicatively coupled with the physiological parameter detection device for receiving and then forwarding the physiological parameter signal;
a comparison and storage module, storing an audio-comparing table of physiological parameters, further including a plurality of audio information; and
a first processing module, electrically coupled with the first transmission module and the comparison and storage module, being to receive the physiological parameter signal so as thereby to locate at least one of the plurality of the audio information in the audio-comparing table of physiological parameters stored in the comparison and storage module that matches the at least one physiological parameter of the user and thus further to generate and then forward an audio signal;
a communication device, further comprising a second transmission module communicatively coupled with the first transmission module of the audio information comparison device for receiving and then forwarding the audio signal; and
an audio device for receiving the audio signal, further comprising a third transmission module and an audio module, the third transmission module being communicatively coupled with the second transmission module of the communication device to receive and then forward the audio signal, the audio module being electrically coupled with the third transmission module to receive the audio signal and then play the corresponding audio information.

2. The hypnotic system of claim 1, wherein the first processing module of the audio information comparison device judges the physiological parameter to determine a sleep cycle information corresponding to the user so as thereby to locate the audio information that matches the physiological parameter from the audio-comparing table of physiological parameters.

3. The hypnotic system of claim 1, wherein the physiological parameter is one of a brain wave, a heartbeat, a breath and a myoelectricity.

4. The hypnotic system of claim 1, wherein the audio information comparison device is a network server.

5. The hypnotic system of claim 1, wherein the communication device is one of a mobile phone and a tablet computer.

6. The hypnotic system of claim 1, wherein the audio device is one of a earphone and a speaker.

7. A hypnotic method, comprising the steps of:
(a) detecting at least one first physiological parameter of a user so as thereby to generate and then transmit a corresponding first physiological parameter signal;
(b) receiving the first physiological parameter signal so as thereby to locate at least one first audio information matching the at least one first physiological parameter from an audio-comparing table of physiological parameters having a plurality of first audio information and then to generate and transmit a corresponding first audio signal;
(c) receiving the first audio signal and playing the first audio information accordingly;
(d) detecting at least one second physiological parameter of the user so as thereby to generate and then transmit a corresponding second physiological parameter signal;
(e) receiving the second physiological parameter signal and judging a respective sleep cycle information so as thereby to locate at least one second audio information matching the at least one second physiological parameter from the audio-comparing table and then to generate and transmit a corresponding second audio signal; and
(f) receiving the second audio signal and playing the second audio information accordingly.

8. The hypnotic method of claim 7, wherein, in the step (f), the first audio information and the second audio information are displayed simultaneously.
